(19) United States
(12) Patent Application Publication
(10) Pub. No.: US 2005/0021048 A1
(43) Pub. Date: Jan. 27, 2005

(54) Catheter introducer and method of introducing a catheter into the heart

(76) Inventors: Steven Michael Kuhlman, Richland, MI (US); Matthew Crosby Flegal, Kalamazoo, MI (US); Donna Jean Waggoner, Kalamazoo, MI (US)

Correspondence Address: FLYNN, THIEL, BOUTELL & TANIS, P.C., 2026 RAMBLING ROAD, KALAMAZOO, MI, 49008-1699, US

(21) Appl. No.: 10/886,271

(22) Filed: Jul. 7, 2004

Related U.S. Application Data
(60) Provisional application No. 60/485,331, filed on Jul. 7, 2003.

(51) Int. Cl. A61F 11/00

(52) U.S. Cl. 606/108; 606/167

(57) ABSTRACT

A catheter introducer and a method of introducing a catheter into a heart. The catheter introducer includes a front puncturing end and a main body portion which defines an upwardly-opening trough therein. A catheter is placed in piggy-back fashion within the trough, and the puncturing end of the tool is inserted into the heart, so that the catheter is delivered into the heart essentially simultaneously with the piercing of the heart wall. The catheter introducer results in a faster introduction of the catheter, and minimizes blood loss and trauma to the heart.

### FIELD OF THE INVENTION

This invention relates to a catheter introducer and a method of implanting a catheter within an area of the heart, such as the left ventricle.

### BACKGROUND OF THE INVENTION

Mammals, including humans, have a double-circuit heart that functions similar to two separate, side-by-side pumps, each with two chambers. On the right side of the heart, blood travels from the right atrium to the right ventricle, and the right ventricle pumps the blood through the pulmonary artery to the lungs for oxygenation. When the oxygenated blood returns to the heart, the blood enters the left atrium before traveling to the left ventricle where the blood is pumped out again with greater force. The left ventricle forces the blood through the ascending aorta and out to the body. The left ventricle of the heart, whether a human heart or an animal heart, thus can be used as a measuring site for monitoring various heart functions, such as left ventricular pressure. This monitoring can be carried out through the use of a catheter which is implanted within the left ventricle, and then appropriately connected to a sensor which senses the pressure in the catheter and transmits pressure readings. A catheter implanted within the left ventricle can also be used for purposes other than monitoring heart function. For example, the implanted catheter can be used to administer drugs directly into the left ventricle, can be used as a vascular access port, can be used for blood collection, or can be used as a port for introduction of a device into the heart, such as, for example, a fiber-optic camera or pressure sensitive crystal. In some cases, the catheter is left in the patient so that heart function can be monitored on a regular basis, or for drug dosing at regular intervals.

The conventional method for introducing a catheter directly through the muscle wall of the heart of an animal, such as a canine, is achieved through the use of suitable scissors, such as iris scissors, or a scalpel blade which incises through the heart wall at the thinnest portion of the heart wall in the left ventricle, known as the “dimple”. Forceps, such as mosquito hemostatic forceps, are then used to hold the stab incision in the left ventricular wall open to allow introduction of the catheter into the heart. This method thus necessarily involves several steps, which complicates and lengthens the time required for the procedure. Further, because a large hole or slit is initially created in the heart muscle, this conventional method can result in significant blood loss and trauma to the heart.

The present invention is directed to a catheter introducer and a method of introducing a catheter directly into the heart, which overcomes the disadvantages of the conventional method described above. More specifically, the catheter introducer according to the invention is an elongated tool having a puncturing or piercing end and a trough-shaped main body portion. The main body portion defines a sidewardly-opening elongated channel or trough therein. A conventional catheter is placed in piggy-back fashion within the trough, and the puncturing end of the tool is inserted into the heart until the tool has penetrated the heart sufficiently to deliver the tip of the catheter into a targeted area of the heart, such as the left ventricle. While the catheter is held in place, the introducer or tool is then withdrawn from the heart, leaving the catheter in place. Sutures are utilized to secure the catheter in position.

The catheter introducer pursuant to the invention allows the catheter to be implanted within the heart wall substantially simultaneously while the opening in the heart wall is being made by the puncturing end of the tool, thereby reducing the amount of time for the procedure. Further, the introducer tool and the catheter carried thereon together define a solid, rod-like configuration which effectively plugs the opening made by the puncturing end of the introducer during the procedure so as to minimize blood loss and cause far less trauma to the heart. Trauma is minimized because the concentric rings of muscle fibers are stretched as they are displaced by the smooth sides of the tool rather than cut or torn.

Other objects and purposes of the invention will be apparent to persons familiar with apparatus of this type upon reading the following description and inspecting the accompanying drawings.

### BRIEF DESCRIPTION OF THE DRAWINGS

FIG. 1 is an elevational and fragmentary side view of the catheter introducer according to the invention.

FIG. 2 is a plan view of the catheter introducer.

FIG. 3 is a longitudinal cross-sectional view of the catheter introducer taken generally along line 3-3 in FIG. 2.

FIG. 3A is an enlarged, transverse cross-sectional view of the catheter introducer taken generally along line 3A-3A of FIG. 3.

FIG. 4 is a perspective view of a canine heart primarily showing the left ventricle.

FIG. 5 is an enlarged, fragmentary view of the apex of the left ventricle shown in FIG. 4.

FIG. 6 is an enlarged, fragmentary cross-sectional view of the apex of the left ventricle of FIG. 4, with the catheter introducer inserted into the muscle wall of the left ventricle.

FIG. 7 is an enlarged, fragmentary view similar to FIG. 5, with the catheter in place in the left ventricle.

Certain terminology will be used in the following description for convenience in reference only, and will not be limiting. For example, the words “upwardly”, “downwardly”, “rightwardly” and “leftwardly” will refer to directions in the drawings to which reference is made. The words “inwardly” and “outwardly” will refer to directions towards and away from, respectively, the geometric center of the device and designated parts thereof. This terminology will include the words specifically mentioned, derivatives thereof, and words of similar import.

### DETAILED DESCRIPTION

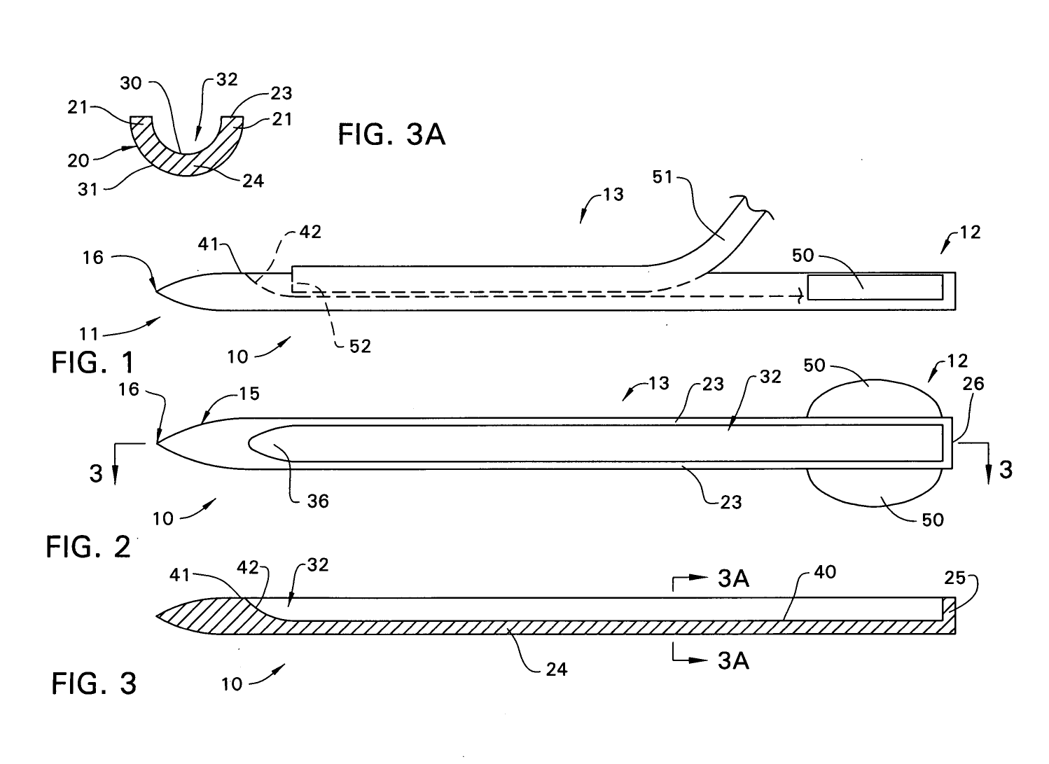

Referring to FIGS. 1-3A, there is illustrated a catheter introducer or tool 10 according to the invention. The catheter introducer generally includes a front puncturing end 11, a rear manipulating end 12, and a main or central body portion 13 disposed between and connected to front and rear ends 11 and 12.

Puncturing end 11 of introducer 10 is defined by a solid and generally bullet-shaped nose 15 which defines a pointed tip 16 at a front end thereof. The outer surface of nose 15 is smooth along its circumference and tapers gradually outwardly as same projects rearwardly from tip 16 towards end 12.

The main body portion 13 projects rearwardly from puncturing end 11 and is defined by a generally arcuate wall 20 (FIG. 3A). Wall 20 includes a pair of side walls 21 which respectively terminate in upper flat edges 23 which extend along a substantial extent of the longitudinal length of introducer 10, and a lower wall 24 which smoothly adjoins the respective side walls 21. A generally upright rear wall 25 which forms part of rear end 12 extends transversely between and is joined to rear edges of the respective side walls 21 and a rear edge of lower wall 24. Rear wall 25 defines an upper surface 26 which adjoins to and is flush with surfaces 21. Arcuate wall 20 defines upper and lower oppositely facing surfaces 30 and 31.

The central body portion 13, through the shape of its arcuate wall 20, defines an upwardly-opening, elongate and arcuate trough or channel 32. In the illustrated embodiment, trough 32 extends along a substantial portion of the longitudinal length of the introducer 10, starting at a point which is located rearwardly from tip 16 and terminating at rear wall 25. Trough 32 has a terminal front end 36 which in the illustrated embodiment has the shape of a partial ellipse when viewed from above.

Trough 32 has a bottom surface 40 which is defined by upper surface 30 of arcuate wall 20. As bottom surface 40 of trough 32 projects forwardly toward puncturing end 11, bottom surface 40 (when viewed in cross-section as in FIG. 3) curves smoothly upwardly to a point which is at the same height as a rear upper surface 41 of nose 15. This configuration of bottom surface 40 provides front end 36 of trough 32 with a surface 42 which curves forwardly as same projects upwardly.

The rear end 12 of introducer 10 mounts thereon a pair of sidewardly projecting and generally horizontally-oriented wings 50 or handles which assist in manual manipulation of introducer 10. In the illustrated embodiment, wings 50 are oriented at the same vertical height as one another on rear end 12, and have a generally semicircular outer edge as shown in FIG. 2. It will be appreciated that other configurations of wings 50 are within the scope of the invention, and that the curved shape shown herein is presented only as an example of one possible configuration of wings 50.

As shown in FIG. 1, the trough 32 is configured to receive a conventional catheter 51. In this regard, trough 32 preferably has a depth which allows approximately three-quarters of the diameter of the catheter 51 to be submerged in trough 32 when positioned on bottom surface 40.

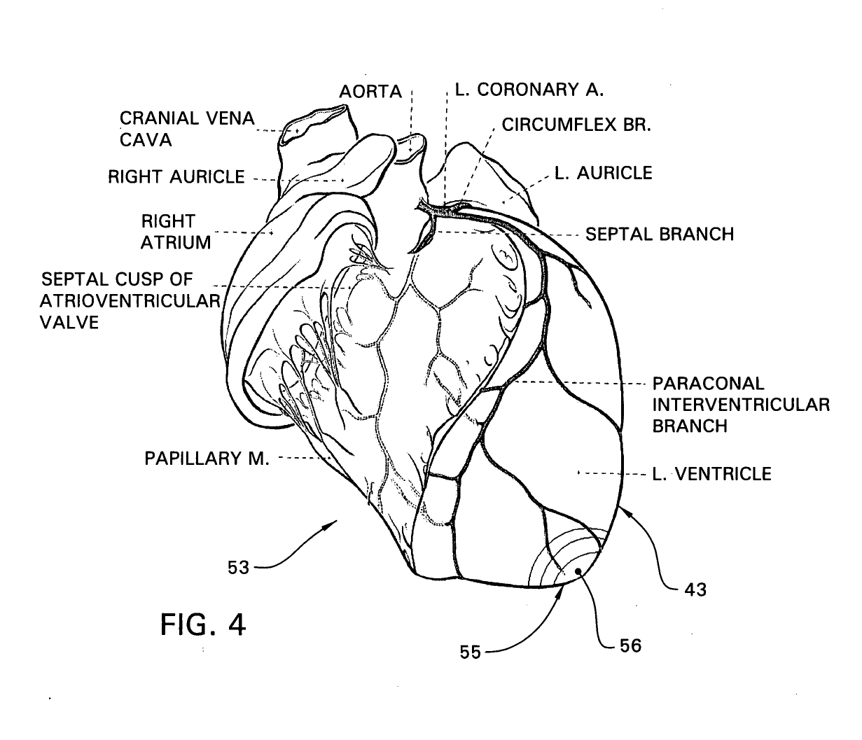

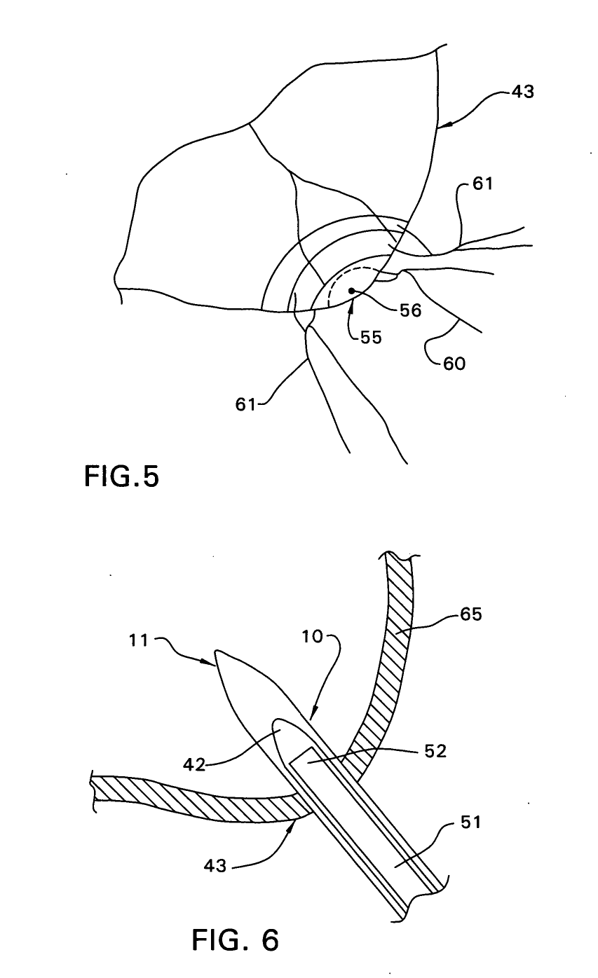

FIG. 4 depicts a heart 53 of an animal, and in the illustrated embodiment, heart 53 is a canine heart. The heart 53 in FIG. 4 is positioned so as to primarily show the left ventricle 54. The left ventricle 54 defines an apex 55 at a lower portion thereof, and a dimple 56 which represents an area of the apex 55 where concentric muscles (schematically represented by concentric lines in FIGS. 4 and 5) of the heart 53 join to one another.

The catheter 51 is introduced or implanted within the heart 53 utilizing introducer or tool 10 according to the invention as follows. Entry into the canine is made by one of two conventional approaches, either a thoracic approach or an abdominal approach in order to expose the apex of the heart 53. The pericardium is incised to free up the apex 55 of the heart 53. A circular purse string-style suture 60 is placed around the dimple 56, which dimple 56 defines the targeted penetration area of the left ventricle 54. Stay sutures 61 are placed on opposite sides of the dimple 56 to assist in holding the heart 53 in the proper position within the chest cavity of the canine.

Next, the tool 10 is positioned so that trough 32 opens generally upwardly, and the catheter 51 is positioned inside the trough 32 so that a frontmost end 52 thereof is located slightly rearwardly from the curved surface 42, with the catheter 51 resting on the bottom surface 40 of trough 32. The front puncturing end 11 of introducer 10 is positioned at the dimple 56, and, using the stay sutures 61 to hold the heart in position, the introducer 10 is pushed into the wall 65 of the left ventricle 54 to pierce same and make an opening. The introducer 10 is pushed into the left ventricle wall 65 until the catheter 51 has penetrated the heart wall 65 and projects into the left ventricle 54 as shown in FIG. 6. The catheter 51 is then held in place with the fingers or with forceps, and the introducer 10 is withdrawn from the heart wall 65, leaving the catheter 51 in place in the heart 53.

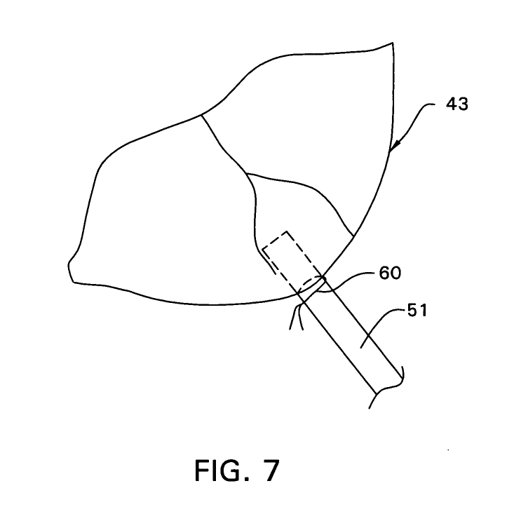

It will be appreciated that the curved surface 42 of trough 32 acts as a ramp, whereby as the introducer 10 is retracted and moved rearwardly away from the catheter 51 and the heart 53, the frontmost end 52 of catheter 51 slides upwardly along surface 42 to assist in removal of introducer 10 from the heart 53. After withdrawal of the introducer 10 from the heart 53, the purse string-style suture 60 is tightened or cinched around the perimeter of the catheter 51 and tied tightly therearound, and is then tied to the catheter 51 itself, as shown in FIG. 7. The stay sutures 61 are removed, and the chest cavity is closed. It may be desirable or necessary to place an additional stay suture (not shown) in the heart wall 65 to the side of the dimple 56, and this stay suture may also be tied around the perimeter of the catheter 51 to provide additional securement of the catheter 51 to the heart wall 65.

The introducer 10 according to the invention effectively punctures the heart wall 65 at the dimple 56, and creates a small, pin prick-type hole in the heart wall 65 which is much smaller than the opening through which the catheter 51 will ultimately extend into the left ventricle 54, and the concentric muscles of the heart wall 65 are stretched open by the introducer 10 and catheter 51. The stretched heart wall 65 adjacent the dimple 56 will then snap into a closed configuration around the catheter 51 when the introducer 10 is removed, and even during the procedure will constrict around the introducer 10 and catheter 51, which minimizes blood loss and trauma to the heart.

The introducer 10 also permits introduction of the catheter 51 into the heart 53 essentially while the opening in the heart 53 is being made, which results in a fastener introduction. Further, the introducer 10 and the catheter 51 carried therein together define a solid and essentially rod-shaped mass which fills or plugs the opening made by the introducer 10, which also minimizes blood loss during the procedure. The smoothly-curved outer surface of nose 15 of introducer 10 also assists in penetrating the heart wall 56.

The catheter 51 once implanted within the heart 53 may be left in place only temporarily, and then removed after desired testing or drug dosing is carried out. Alternatively, the catheter 51 may be left in place permanently or for a set period of time, for example when it is desirable or necessary to carry out drug dosing at regular intervals or when it is desirable to monitor functioning of the heart 53 on a regular basis. If the catheter 51 is intended for permanent placement in the heart 53, the outer end of the catheter 51 can be connected to an appropriate sensor or transmitter, such as a piezo-electric sensor (not shown) which may be provided subcutaneously and from which various readings can be obtained which relate to heart function. Alternatively, the outer end of the catheter 51 may exit the body of the animal and can be suitably closed off by a cap which seals the end of the catheter to the environment but is penetrable to allow drug dosing.

It will be appreciated that the introducer 10 according to the invention may be utilized with animals other than canines, for example, guinea pigs, rabbits, etc., and can accordingly be scaled up or down so as to accommodate the particular animal and the size of catheter being introduced. It is contemplated that the introducer 10 pursuant to the invention may also be utilized in humans.

It will be understood that the introducer 10 may be utilized to implant a catheter or other device within areas of the heart other than the left ventricle, and the illustrations and description herein relating to the left ventricle are presented as only an example of one usage of the introducer.

Referring back to FIGS. 1-3A, it is to be understood that the trough 32 need not extend along the entire longitudinal extent of the introducer 10, and instead may terminate forwardly of rear wall 25.

The introducer 10 according to the invention may be constructed of rigid metal, such as stainless steel.

Although a particular preferred embodiment of the invention is disclosed in detail for illustrative purposes, it will be recognized that variations or modifications of the disclosed apparatus, including the rearrangement of parts, lie within the scope of the present invention.

1. An apparatus for introducing a catheter into the heart, said apparatus comprising an elongate main body portion having an end portion defining a pointed configuration for piercing a wall of the heart, said main body portion defining therein a sidewardly opening trough for receiving therein a terminal end portion of a catheter to be introduced into the heart through a wall thereof.

2. The apparatus of claim 1 wherein said end portion is a first end portion and said main body portion has a second end portion defining a pair of sidewardly projecting wings thereon for assisting in manual manipulation of said apparatus by a user.

3. The apparatus of claim 1 wherein said main body portion includes a bottom wall which defines a bottom of said trough, said trough adjacent said end portion has an arcuate portion which is smoothly joined to said bottom wall and curves towards said end portion as same projects upwardly from said bottom wall, said arcuate portion permitting removal of the catheter from said apparatus after insertion of the catheter into the heart.

4. The apparatus of claim 1 wherein said main body portion is defined by an elongate wall which is arcuate in transverse cross-section.

5. The apparatus of claim 1 wherein said trough terminates at a front end which is positioned closely adjacent and rearwardly of said end portion of said main body portion, and the end portion of the catheter is positioned within said front end portion such that the catheter end portion is introduced into the heart substantially simultaneously with the puncturing of the wall of the heart by said end portion.

6. A catheter introducer comprising a main body portion having one end configured for puncturing a wall of a heart, said main body portion defining therein a channel within which a catheter is positionable for introduction into the heart.

7. The catheter introducer of claim 6 wherein said channel opens sidewardly to allow positioning of the catheter therein.

8. The catheter introducer of claim 6 wherein an opposite end of said main body portion defines a handle thereon for manipulation of said introducer.

9. The catheter introducer of claim 6 wherein an end of said channel is positioned closely adjacent said one end such that the catheter is introduced into the heart substantially simultaneously with the puncturing of the heart wall by said one end.

10. The catheter introducer of claim 6 wherein said channel opens sidewardly along a length thereof, said channel having a front end portion having a configuration which curves gradually upwardly and frontwardly from a bottom of said channel and adjoins an upper surface of said one end of said main body portion.

11. The catheter introducer of claim 10 wherein the catheter has an end portion positioned within said front end portion of said channel, and said configuration of said front end portion permits withdrawal of said introducer away from the catheter after insertion of the catheter into the heart.

12. A method of introducing a catheter into a heart, said method comprising the steps of:
providing a tool having a piercing end and a main body portion connected to the piercing end, said main body portion defining a trough therein;
providing a catheter;
positioning an end portion of the catheter within the trough of the tool;
puncturing a wall of the heart with the piercing end of the tool to create an opening in the wall;
inserting the tool and the end portion of the catheter positioned therein through the opening and into the heart; and
withdrawing the tool from the heart and away from the catheter so that the end portion thereof is positioned within the heart.

13. The method of claim 12 wherein said step of positioning is performed prior to said step of puncturing.

14. The method of claim 12 wherein said steps of puncturing and inserting are performed substantially simultaneously with one another.

15. The method of claim 12 wherein said step of withdrawing includes holding the catheter in position within the heart while the tool is being withdrawn therefrom.

16. The method of claim 12 including the step of securing the catheter within the heart with sutures.

17. The method of claim 12 including introducing the catheter into the left ventricle of a canine heart.

18. The method of claim 12 wherein said step of puncturing includes puncturing a wall of the left ventricle with the piercing end of the tool to create an opening in the left ventricle wall.

19. The method of claim 18 wherein said step of inserting includes inserting the tool and the end portion of the catheter positioned therein through the opening and into the left ventricle.

20. The method of claim 19 wherein said step of withdrawing includes withdrawing the tool from the left ventricle and away from the catheter so that the end portion thereof is positioned within the left ventricle.
